(19) United States
(12) Patent Application Publication
(10) Pub. No.: US 2007/0238780 A1
(43) Pub. Date: Oct. 11, 2007

(54) Multifunctional enhancement systems for personal care, household, and wood products

(76) Inventor: Patrick Jay Lutz, Nazareth, PA (US)

Correspondence Address: Irving M. Fishman, Suite 1422, North Tower, 89 Headquarters Plaza, Morristown, NJ, 07960, US

(21) Appl. No.: 11/358,521

(22) Filed: Feb. 21, 2006

Related U.S. Application Data
(60) Provisional application No. 60/655,212, filed on Feb. 22, 2005.

(51) Int. Cl. C07C 233/00; A01N 33/00; C07C 53/00; C07D 309/30; C11D 3/48; C07D 277/02; A01N 37/00

(52) U.S. Cl. 514/506; 510/382; 514/1; 514/578; 514/663; 548/146; 549/292; 554/35

(57) ABSTRACT

A personal care product, a household product, an industrial formulation, or a medicinal product of at least a fluorosurfactant and/or a natural or naturally-derived or synthetic, and/or *Eco-friendly (See Description) materials such as PEG-4 rapeseedamide, glycerth 2-cocate, Behenamidopropyl dimethylamine, and lactic acid (and derivatives of) as components and at least a second component (which may or may not be present in the formulation in question already) selected from the group consisting of citric acid, glyconic acid, gluconic acid, sodium erythorbate, iodo propynyl butyl carbamate, antioxidants, dehydroacetic acid, parabens, DMDM hydantoin, diazolidinyl urea, caprylyl glycol, glucono Δ lactone, EDTA, amine oxides (other than fluorosurfactants having amine oxide groups, which are included as component 1 materials), glutaraldehydes, chlorhexidine, phenoxyethanol, benzyl alcohol, salicylic acid, dipropylene glycol, quaternary ammonium (benzalkonium and benzethonium chlorides), glycereth-2-cocoate, PEG-4 Rapeseedamide, silver methyl-dibromo-glutaronitrile, sodium hydroxymethylglycinate, methylchloroisothiazolinone, methyl isothiazolinone, chlorophenesin, sodium sulfite, triclosan, carboxylic acid (adipic acid), benzoic acid, sorbic acid, hyaluronic acid, alpha lipoic acid, erythorbic and any of the salt forms thereof. Product improving premixes of the fluorosurfactant and the second component for addition to and improve of other products are also claimed.

### CROSS-REFERENCE TO RELATED APPLICATIONS

Not applicable.

### STATEMENT REGARDING FEDERALLY SPONSORED RESEARCH OR DEVELOPMENT

Not Applicable

### FIELD OF THE INVENTION

The present invention relates to combination systems that lower surface tension and increase the effectiveness and stability of other actives contained in the compositions to which the present systems are added. More specifically, the present invention relates to a binary, tertiary or quaternary (and more complex mixture) component system having at least one of a fluorosurfactant and/or a natural or naturally-derived or synthetic, and/or *Eco-friendly materials such as PEG-4 rapeseedamide, glycerth-2-cocoate, behenamidopropyl dimethylamine, (and derivatives of) as components. The invention systems further relate to stability improvements in a wide range of consumer products, including, but not limited to personal care and household, industrial and institutional products, as well as pool and wood products, having a wide range of active components therein. In certain embodiments, the invention especially relates to cleaners and disinfectants. Still further, the present invention relates to medicinal products, particularly those that are topical. *Eco-friendly products are products that are tested by independent third parties for safer toxicology profiles for the environment. Typically these products have a higher biodegradability and low aquatic toxicology than other alternatives. Such Eco-friendly products include, but are not limited to, glycereth 2 cocoate, PEG-4 rapeseedamide, sodium laureth sulfate, sodium Laureth-11 carboxylate+laureth-10, behenoyl PG-trimonium chloride, and behenamidopropyl dimethylamine. Additional Eco-friendly materials and products can be found at www.europa.eu.int/comm/environment/ecolabel, and such list is incorporated herein by reference.

### BACKGROUND OF THE INVENTION

In many products, especially personal care products, there are no multifunctional systems that lower surface tension and simultaneously improve the effectiveness and stability of the formulation and particularly the other active components present in the products to which they are added. Typically, surfactants are added to systems with a desire to simply increase wettability of compositions intended to be delivered, or, as in cleaners, to increase the ability of the composition to wet a surface of material that is intended to be removed. Little or no thought is given to the surfactant in terms of increasing the inherent ability of the active agent to perform its function better or making cell membranes more permeable for the improved performance. Also there are no stable natural and/or naturally-derived systems that are water-white liquid systems and have synergistic broad spectrum activity.

Most cleansers, whether personal care products, household products, and even industrial products, include surfactants merely as a basis for dissolving or emulsifying the soils and stains to be removed. Most typically these surfactants are not of fluorinated materials but are of the polyethoxylated type, fatty alcohol sulfate ester type, and/or cationic salts of fatty acid. While the inclusion of these typical surfactants may be suitable for aiding in washing away materials and for creating various emulsion systems as delivery vehicles, there is still much in the way of improved systems that are needed in this field.

Moisturizers and sunscreen formulations are other examples where the currently available products are insufficient. Many moisturizers and sunscreen compositions have complex mixtures of materials that resist spreading and penetration into the skin. The typical surfactant systems in such products are also those non-fluorinated surfactants mentioned above. Similar issues are present in many medicinal products, especially those that are topical in nature.

There is thus a need to improve the surfactant properties of a wide range of products that are currently in the stream of commerce.

### OBJECTS OF THE INVENTION

It is therefore an object of the invention to provide a multifunctional system that is suitable for incorporating into a product where the multifunctional system lowers the surface tension and increases the stability and/or effectiveness of the active agents present.

It is a further object of the invention to provide stable natural and/or naturally-derived systems that are water-white liquid systems and have synergistic broad spectrum activity.

It is another object of the invention to provide an improved personal care product.

It is still another object of the invention to provide an improved industrial product.

It is another object of the invention to provide an improved medicinal product.

It is a further object of the invention to provide a topically applied medicinal or personal care product demonstrating improved leveling and/or spreading.

It is an even further object of the invention to provide a product having improved bacteriocidal and/or enhanced bacteriostatic properties.

It is an even further object of the invention to provide a product having improved the stability of concentrated multifunctional systems.

It is yet another object of the invention to provide an improved biofilm penetration product.

It is an even further object of the invention to provide products having improved solubility of difficult to dissolve actives such as IPBC, parabens, dehydroacetic acid, natural actives etc.

It is still another object of the invention to provide a method of achieving the foregoing objects of the invention by inclusion of at least one of a fluorosurfactant and/or a natural or naturally-derived PEG-4 rapeseedamide and/or glycereth 2 cocoate into the respective formulations.

Still further objects of the invention will be apparent to those of ordinary skill in the art.

### BRIEF SUMMARY OF THE INVENTION

These and other objects of the invention are surprisingly achieved by the inclusion into a personal care formulation, a household product formulation, an industrial formulation, or a medicinal product formulation of at least a first component selected from the group consisting of a fluorosurfactant, a natural or naturally-derived component such as PEG-4 rapeseedamide, glycereth 2 cocoate, and behenamidopropyldimethylamine and mixtures thereof and at least a second component (which may or may not be present in the formulation in question already) selected from the group consisting of carboxylic acids (such as, without limitation, adipic acid, alpha lipoic acid, amino acids (such as without limitation, L-lysine and taurine), benzoic acid, citric acid, dehydroacetic acid, EDTA, erythorbic acid, glycolic acid, glyconic acid, gluconic acid, hyaluronic acid, lactic acid, pyrrolidone carboxylic acid, salicylic acid, sodium erythorbate, sorbic acid, and any of the salt forms thereof), iodo propynyl butyl carbamate, antioxidants (including, but not limited to BHA and BHT), dehydroacetic acid, parabens, formaldehyde donors (such as DMDM hydantoin), diazolidinyl urea, caprylyl glycol, glucono Δ lactone, amine oxides (other than fluorosurfactants having amine oxide groups, which are included as component 1 materials), glutaraldehydes, chlorhexidine, alcohols such as phenoxyethanol and benzyl alcohol, dipropylene glycol, quaternary ammonium (benzalkonium and benzethonium chlorides), silver methyl-dibromo-glutaronitrile, sodium hydroxymethylglycinate, methylchloroisothiazolinone, methyl isothiazolinone, chlorophenesin, sodium sulfite, triclosan, citrus terpenes, aliphatic dibrominated diol (such as 2-bromo-2-nitropropane-1,3-diol), and quaternary ammonium compounds (such as without limitation, benzalkonium chloride and benzethonium chloride). If the first component is already present in a formulation, then the addition of the second component achieves the objects of the invention. If the second component is already present in a formulation, then the fluorosurfactant and/or PEG-4 rapeseedamide and/or glycereth 2 cocoate being added thereto achieve the objects of the invention. Otherwise, both the first and second components are added, either separately or together. In preferred embodiments, formulations of fluorosurfactants and/or PEG-4 rapeseedamide and/or glycereth 2 cocoate with one or more of the second components are preformulated and added as a fixed combination, although additional amounts of either the first and/or second components can be added as desired as well.

### BRIEF DESCRIPTION OF THE DRAWING

Not applicable

### DETAILED DESCRIPTION OF THE INVENTION

The present invention is directed to a formulation containing at least a first component selected from the group consisting of one or more of a fluorosurfactant and/or a natural or naturally-derived pegylated rapeseedaminde having 2- about 8 ethoxy groups per molecule (preferably 3-4 ethoxy groups per molecule, more preferably PEG-4 rapeseedamide) and/or glycereth-2-fatty acid ester having 8-16 carbons in the fatty acid portion (which fatty acid may be straight or branched, saturated or unsaturated, preferably having about 12 carbons in the fatty acid portion, also preferably the fatty acid portion is straight chain, and also preferably the fatty acid portion is saturated, more preferably the glycereth-2-fatty acid ester is glycereth-2-cocoate), and/or a C₁₆-C₂₄fatty acid-amido-C₁-C₆alkylene-di(C₁-C₄ alkyl)amine (the fatty acid portion being straight chain or branched, saturated or unsaturated; preferably straight chain and preferably saturated, and preferably being about 18-22 carbons, most preferably being behenoyl; the alkylene portion being straight chain or branched, preferably straight chain and preferably having 2-4 carbon atoms, more preferably propylene, and the alkyl groups in the dialkylamino group may be the same of different, preferably the same, and preferably are methyl, most preferably behenoylamidopropyldimethylamine), and mixtures thereof; and a second component chosen from a select group of materials as detailed more fully below to give a first product. The first product is a product that enhances not only the reduction in surface tension of the formulation to which it is added, but results in improved effectiveness of many of the active agents contained in the formulations to which the first product is added. It also results in improved stability of the product to which it is added. In situations where one of the first or second component is already present in a given formulation with other actives, the addition of the other component will achieve the desired results of the present invention and is deemed to part of the present invention.

The first component fluorosurfactant means a poly fluoroalkyl group bound to a solubilizing head, usually through an alkylene bridge. Preferably the polyfluoroalkyl group is perfluoroalkyl. While any length of the fluorinated alkyl group is suitable, carbon chains in the fluorinated portion of the molecule are generally at least 4 carbons in length, preferably 4-18 carbons in length. Individually preferred ranges for fluorinated alkyl length include without limitation, 4-16 carbons, 8-18 carbons, and 4-18 carbons. Generally a particular fluorosurfactant is a mixture of materials having different carbon lengths in the fluoroalkyl portion, with most, if not all of the fluorosurfactant having fluoroalkyl groups within the stated individually preferred ranges. However, purified individual compounds with a very particular fluoroalkyl carbon length are also within the definition of the present invention fluorosurfactants. Thus the fluoroalkyl groups are generally of the formula:
CHₐF_{b}(CH_{c}F_{d})ₓ—
where each of a and b is 0-3, a+b is 3, each of c and d is 0-2, c+d is 2, wherein a substantial number of the carbon atoms have at least one fluorine atom thereon. Preferably a and c (in all repeating units) are both zero, b is 3 and d (in all repeating units) is 2 (i.e. a perfluoroalkyl).

The fluorinated alkyl group is generally bound to the solubilizing head through an alkylene or heteroatom interrupted alkylene bridge, although the bridge may be absent. Thus, the fluorosurfactant is generally of the structure:
fluoroalkyl-((alkylene-(interrupting heteroatom)ₚ)_{q}-alkylene)ᵣ-solubilizing head
where p is 0 or 1, preferably 0, q may be 0-4, but must be zero when p is zero, and r is zero or 1. The alkylene groups within ((alkylene-(interrupting heteroatom)ₚ)_{q}-alkylene)ᵣ are each independently from 1 to 4 carbons in length, preferably 1-3 carbons in length, most preferably about 2 carbons in length. The alkylene bridge may be substituted with other lower alkyl groups, typically of 1-4 carbons, preferably of 1-3 carbons, more preferably methyl or ethyl, but most preferably the alkylene bridge is unsubstituted. Interrupting heteroatoms include O, S, N, and such nitrogens can carry further lower alkyl or fluoroalkyl groups.

The solubilizing head portion of the fluorosurfactant includes phosphate esters (—OP(O)(OH)₂); betains (—N⁺(lower alkyl)₂—CH₂C(O)O⁻—) and the related sultains; tri(lower alkyl ammonium); di-lower alkyl amine oxides; diesters of sulfosuccinate (alkali metal salt); carboxy (—C(O)O⁻); polyalkylene oxide ((—OC₂₋₃alkyl)ₜ—OH) (with t being from about 2 to about 20; and fatty acid esters (—O(O)C—C₈₋₂₄saturated or unsaturated aliphatic). In each of the above solubilizing head portions, “lower alkyl” is independently of 1-4 carbons, preferably 1-3 carbons, more preferably methyl or ethyl, most preferably methyl.

A highly preferred non-limiting set of fluorosurfactants suitable for use in the present invention are the following materials, all available from Mason Chemical Company: MASURF FS-130; MASURF FS-115; MASURF FS-130; MASURF FS-330; MASURF FS-230; MASURF FS-330; MASURF FS-1520; MASURF FS-1620; MASURF 130A; MASURF FS-615; MASURF FS-710; MASURF FS-780; MASURF FS-1030; MASURF FS-1230; MASURF FS-1400; MASURF FS-1520; MASURF FS 1725; MASURF FS-1700; MASURF FS-1825; MASURF FS-1800; among others.

The naturally-derived PEG-4 rapeseedamide, is without limitation, frequently derived from rapeseed oil. Other sources will be known to those of ordinary skill in the art.

The second component of the invention is selected from a group consisting of iodo propynyl butyl carbamate, gluconic acid, citric acid, parabens (inclusive of methyl paraben, ethyl paraben, propyl paraben, and benzyl paraben), antioxidants (such as butylated hydroxyl anisole (BHA), BHT, et cet.), alcohols (such as phenoxyethanol), methyl-dibromo glutaronitrile, sodium hydroxymethyl glycinate, aliphatic dibrominated diol (such as 2-bromo-2-nitropropane-1,3-diol), methylchlorothiazolidinone, methylisothiazolidinone, chlorophenesin, sodium sulfite, salicyclic acid, triclosan, organic acids/salts (such as dehydroacetic acid, salicylic acid, benzoic acid and sorbic acid and/or salts thereof, natural or naturally derived actives (such as glucono-delta-lactone, gluconic acid, glycolic acid, alpha lipoic acids, and hyaluronic acids), formaldehyde donors such as DMDM hydantoin, sodium erythorbate, quaternary ammonium compounds (such as alkyl or aryl alkonium halides; betains, or sultaines)(where the alkyl groups can be from lower alkyl of as little as 1 carbon up to fatty alkyl up to 20 or more carbons), and mixtures thereof.

Specific preferred combinations can be found in the table below, however, this table is merely exemplary and does not limit the scope of the invention.

| Component 1a | Component 1b | Component 2 |
|---|---|---|
| Fluorosurfactant | PEG-4 Rapeseedamide | |
| Fluorosurfactant | Glycereth-2 cocoate | |
| Fluorosurfactant | | Sodium PCA* |
| Fluorosurfactant | | Lauroyl Lysine |
| Fluorosurfactant | | Benzoic acid or salt form |
| Fluorosurfactant | | Lactic acid or salt form |
| Fluorosurfactant | | IPBC |
| Fluorosurfactant | | DMDM Hydantoin |
| Fluorosurfactant | | Glucono Delta Lactone |
| Fluorosurfactant | | Gluconic acid |
| Fluorosurfactant | PEG-4 Rapeseedamide | Lactic acid or salt form |
| Fluorosurfactant | Glycereth-2 cocoate | Benzoic acid or salt form |
| Glycereth-2 Cocoate | | Lactic + Benzoic acids |
| Glycereth-2 Cocoate | | Sodium PCA* |
| Glycereth-2 Cocoate | | Lauroyl Lysine |
| Glycereth-2 Cocoate | | Sodium PCA* + Benzoic |
| | | acid or salt form |
| Glycereth-2 Cocoate | | Lauroyl Lysine + |
| | | Benzoic acid or salt form |
| PEG-4 Rapeseedamide | | Citrus Terpenes |
| PEG-4 Rapeseedamide | | Lactic + Benzoic acids |
| Behenamidopropyl | | Lactic + Benzoic acids |
| Dimethylamine | | |

*PCA = sodium pyrrolidone carboxylic acid

Other combinations that are particularly preferred include either one, two, three or four of the fluorosurfactant, Glycereth-2 Cocoate, PEG-4 Rapeseedamide and/or behenamidopropyldimethylamine as component 1 and one or two of the following as component 2:

carboxylic acids (such as, without limitation, adipic acid, alpha lipoic acid, amino acids (such as without limitation, L-lysine and taurine), benzoic acid, citric acid, dehydroacetic acid, EDTA, erythorbic acid, glycolic acid, glyconic acid, gluconic acid, hyaluronic acid, lactic acid, pyrrolidone carboxylic acid, salicylic acid, sodium erythorbate, sorbic acid, and any of the salt forms thereof), iodo propynyl butyl carbamate, antioxidants (including, but not limited to BHA and BHT), dehydroacetic acid, parabens, formaldehyde donors such as DMDM hydantoin, diazolidinyl urea, caprylyl glycol, glucono Δ lactone, amine oxides (other than fluorosurfactants having amine oxide groups, which are included as component 1 materials), glutaraldehydes, chlorhexidine, alcohols such as phenoxyethanol and benzyl alcohol, dipropylene glycol, quaternary ammonium (benzalkonium and benzethonium chlorides), silver methyl-dibromo-glutaronitrile, sodium hydroxymethylglycinate, methylchloroisothiazolinone, methyl isothiazolinone, chlorophenesin, sodium sulfite, triclosan, citrus terpenes, aliphatic dibrominated diol (such as 2-bromo-2-nitropropane-1,3-diol), and quaternary ammonium compounds (such as benzalkonium chloride and benzethonium chloride).

These combinations of materials are particularly advantageous (in providing the benefits of the invention, although the full scope of the invention is set forth in the full specification and claims). When added to antimicrobial compositions, such as personal care cleansers, household cleansers, industrial cleansers that have antimicrobial components therein, the antimicrobial efficacy is enhanced. The surface tension of the cell membrane of the microbial cell and the surrounding environment is reduced, thereby making the cell membrane more permeable and accessible to the antimicrobial active. Transfer across the water-filled trans-membrane diffusion channels is also enhanced. Thus, the component 1 materials have antimicrobial properties of their own and may also be used alone as the antimicrobial component of an antimicrobial compositions or in combinations with other antimicrobial components in such compositions.

The mixture may be heated and/or stirred to expedite mixing. The concentrate may include from about 0.01 to about 100% by weight of the component 1 active components and preferably contains from about 1 to about 80% by weight of the all component 1 and component 2 active components present therein based upon 100% total weight of concentrate.

Use Dilutions

Use dilutions generally contain from about 0.01% to about 2% by weight of the concentrate, especially where the concentrate has higher proportions of the actives therein. Where the concentrates have lower concentrations of active components, the use dilutions generally may contain higher percentages of the concentrate, as desired. Of course, if weaker or stronger concentrates are used, the use dilution of the concentrate will vary accordingly as can well be appreciated by those of ordinary skill in the art. While dilution of the concentrate is highly desirable, the use of the concentrate itself with minor dilution is also possible. For example, a concentrate may merely have the solid components of a formulation added thereto without further addition of solvent or water so that the product of use has nearly the same concentration of components 1 and 2 as the concentrate. This is especially the case when the concentrates are comprised of a substantial amount of water or solvent.

When the invention combinations are added to moisturizers, they improve the accessibility of the moisturizer to the skin via modification and improvement of a leveling or spreading effect. In addition, the moisturizer is better able to penetrate into the skin.

When added to compositions in general having various preservatives, the combinations of the invention improve the biofilm penetration of the active agents into the biofilms via substantial reduction of the biofilm surface tension.

When added to sunscreen compositions, the invention combinations allow for improved protection by the sunscreen by allowing the sunscreen formulation to more evenly spread and provide maximum sunscreen protection.

### EXAMPLES

The following examples exemplify, but do not limit, the present invention.

### Example 1

### Antibacterial Properties of Component 1 Compounds

Minimum Inhibitory concentration tests are conducted using a Masamide R-4 or a 20% active solution of Masurf FS-1620 (perfluoroalkylethyl thiohydroxypropyl-trimonium chloride, available from Mason Chemical). Each of E. coli, S. aureus, P. aeruginosa, C. albicans, and A. niger are exposed to various concentrations of these materials as set forth below. The results are reported in Table 1.

TABLE 1
Active Levels

| | | | | | | | |
|---|---|---|---|---|---|---|---|
| Organism | 20% | 15% | 12.5% | 10% | 5% | 2.5% | 1.25% |

Masamide R-4 - 90% active (PEG-4 Rapeseedamide)

| | | | | | | | |
|---|---|---|---|---|---|---|---|
| E. coli | Sig. Red. | G | G | G | G | G | G |
| S. aureus | G | G | G | G | G | G | G |
| P. aueruginosa | Sig. Red. | G | G | G | G | G | G |
| C. albicans | Sig. Red. | G | G | G | G | G | G |
| A. niger | G | G | G | G | G | G | G |

Masurf FS-330 -30% active (C8-18 perfluoroalkylethyl betaine)

| | | | | | | | |
|---|---|---|---|---|---|---|---|
| E. coli | Sig. Red. | G | G | G | G | G | G |
| S. aureus | Sig. Red. | G | G | G | G | G | G |
| P. aueruginosa | NG | G | G | G | G | G | G |
| C. albicans | Sig. Red. | G | G | G | G | G | G |
| A. niger | G | G | G | G | G | G | G |

Masurf FS-1620 (Perfluoroalkylethyl thiohydroxypropyl-trimonium chloride) (20% active)

| Organism | 10% | 5% | 2.5% | 1.25% | 1% | 0.5% | 0.25% |
|---|---|---|---|---|---|---|---|
| E. coli | NG | NG | NG | NG | NG | NG | NG |
| S. aureus | NG | NG | NG | NG | NG | NG | NG |
| P. aueruginosa | NG | NG | NG | NG | NG | NG | NG |
| C. albicans | NG | NG | NG | NG | NG | NG | NG |
| A. niger | NG | NG | NG | NG | NG | NG | NG |

Sig. Red. - Significant Reduction
NG = no growth
G = growth

Thus, it is clear that each of the Masamide R-4 and the fluorosufactant has its own antibacterial properties independent of other active agents which may be present in various formulations in which they are to be incorporated into. As such, the component 1 components may be used in place of or to enhance the effectiveness of an antibacterial component in a finished formulation. This is especially advantageous where other antibacterial have incompatibilities with one or more of the other required components of a particular formulation, yet an antibacterial property for such a composition is still desired.

### Example 2

Stability Improvements

Stability testing is conducted by following color changes in particular formulations from a first color when initially prepared through 1 month of storage at 50° C. Particular combinations along with their initial color and their color after being stored for 1 month at 50° C. are set forth in Table 2 below.

TABLE 2

| | | Color on storage |
|---|---|---|
| Compositions | Initial color | for 1 mo. at 50° C. |
| 1% IPBC + 99% Masurf G-2C | water white | gold to dark yellow |
| 1% IPBC + 98% Masurf G-2C + 1% Masurf FS 1620 | water white | light yellow |
| 1% IPBC + 89% Masurf G-2C + 10% Gluconic acid | light yellow | yellow |
| 20% of 10% gluconic acid + 80% DMD Hydantoin | light yellow | yellow |
| 20% of 10% gluconic acid + 79% DMD Hydantoin + | light yellow | light yellow |
| 1% Masurf FS 1620 | | |
| 10% gluconic acid + 90% Masurf G-2C | water white | yellow |
| 10% gluconic acid + 89% Masurf G-2C + | water white | water white |
| 1% Masurf FS 1620 | | |
| 30% GDL + 10% gluconic acid | water white | light yellow |
| 30% GDL + 10% gluconic acid + 4% Masurf FS 1620 | water white | water white |
| 15% Benzoic acid + 85% Masurf G-2C | water white | light amber |
| 15% Benzoic acid + 80% Masurf G-2C + 5% Masurf FS 1620 | water white | water white |
| 30% Citrus Terpenes + 70% Masamide R-4 | light yellow | Dark yellow - gold |
| 30% Citrus Terpenes + 65% Masamide R-4 + 5% FS1620 | light yellow | light yellow |

IPBC = iodo propynyl butyl carbamate
GDL = gluocono- delta lactone.

Thus, it can be readily seen that the present invention component 1 enhances the stability of compositions in which it is incorporated.

### SEQUENCE LISTING

Not Applicable

We claim:

1. A combination comprising a first component selected from at least one member of the group consisting of fluorosurfactants, a PEGylated rapeseedamide, glycerth 2-fatty acid esters, fatty acidamidoalkylenedi(alkyl)amines, and mixtures thereof and a second component selected from the group consisting of iodo propynyl butyl carbamate, gluconic acid, citric acid, parabens, antioxidants, alcohols, methyl-dibromo glutaronitrile, sodium hydroxymethyl glycinate, aliphatic dibrominated diol, methylchlorothiazolidinone, , and mixtures thereof, methylisothiazolidinone, chlorophenesin, sodium sulfite, salicyclic acid, triclosan, organic acids or salts thereof, formaldehyde donors, sodium erythorbate, quaternary ammonium compounds.

2. The combination of claim 1 wherein said fluorosurfactant has a fluoroalkyl group of at least 4 carbons bound to a solubilizing head directly or through an uninterrupted or heteroatom interrupted alkylene bridge; said PEGylated rapeseedamide has about 2 to about 8 ethoxy groups; said glycereth 2-fatty acid ester has a fatty acidy group having which has about 8 to about 16 carbons; and said fatty acidamidoalkylenedi(alkyl)amine is a C₁₆-C₂₄fatty acidamidoC₁-C₆alkylenedi(C₁-C₄alkyl)amine.

3. The combination of claim 1 wherein said fluorosurfactant is of the formula fluoroalkyl-((alkylene-(interrupting heteroatom)ₚ)_{q}-alkylene)ᵣ-solubilizing head I where fluoroalkyl is
CHₐF_{b}(CH_{c}F_{d})ₓ— II
where each of a and b is 0-3, a+b is 3, each of c and d is 0-2, c+d is 2, wherein a substantial number of the carbon atoms have at least one fluorine atom thereon, p is 0 or 1, preferably 0, q may be 0-4, but must be zero when p is zero, and r is zero or 1, the alkylene groups within ((alkylene-(interrupting heteroatom)ₚ)_{q}-alkylene)ᵣ are each independently from 1 to 4 carbons in length, the alkylene groups may be substituted with other lower alkyl groups, interrupting heteroatoms are selected from O, S, N, and such nitrogens can carry further lower alkyl or fluoroalkyl groups, the solubilizing head portion of the fluorosurfactant includes phosphate esters (—OP(O)(OH)₂); betains (—N⁺(lower alkyl)₂—CH₂C(O)O⁻—) and the related sultains; tri(lower alkyl ammonium); di-lower alkyl amine oxides; diesters of sulfosuccinate (alkali metal salt); carboxy (—C(O)O⁻); polyalkylene oxide ((—OC₂₋₃alkyl)ₜ—OH) (with t being from about 2 to about 20), and fatty acid esters (—O(O)C—C₈₋₂₄saturated or unsaturated aliphatic);
said PEGylated rapeseedamide is PEG 4 rapeseedamide;
said glycereth 2 fatty acid ester is glycereth 2-cocoate; and
said fatty acidamidoalkylenedi(alkyl)amine is behenamidopropyl dimethylamine.

4. The combination of claim 1 wherein second component is at least one member selected from the group consisting of
iodo propynyl butyl carbamate, gluconic acid, citric acid, parabens, butylated hydroxyl anisole (BHA), BHT, phenoxyethanol, methyl-dibromo glutaronitrile, sodium hydroxymethyl glycinate, 2-bromo-2-nitropropane-1,3-diol), methylchlorothiazolidinone, methylisothiazolidinone, chlorophenesin, sodium sulfite, salicyclic acid, triclosan, dehydroacetic acid, salicylic acid, benzoic acid and sorbic acid, lactic acid, glucono-delta-lactone, gluconic acid, glycolic acid, alpha lipoic acids, and hyaluronic acids, DMDM hydantoin, sodium erythorbate, alkylalkonium halides, arylalkonium halides; betains, sultaines and salts thereof.

5. A method of enhancing the effectiveness of an active agent in a cleaning product containing an antimicrobial or a preservative comprising incorporating into such product at least a first component said first component comprising at least one member selected from the group consisting of fluorosurfactants, a PEGylated rapeseedamide, glycerth 2-fatty acid esters, fatty acidamidoalkylenedi(alkyl)amines, and mixtures thereof; and
optionally one or more members selected from the group consisting of iodo propynyl butyl carbamate, gluconic acid, citric acid, parabens, antioxidants, alcohols, methyl-dibromo glutaronitrile, sodium hydroxymethyl glycinate, aliphatic dibrominated diol, methylchlorothiazolidinone, , and mixtures thereof, methylisothiazolidinone, chlorophenesin, sodium sulfite, salicyclic acid, triclosan, organic acids or salts thereof, formaldehyde donors, sodium erythorbate, quaternary ammonium compounds

6. A method of improving the effectiveness of an active agent in a cleaning product containing an antimicrobial or a preservative and containing at least one at least one member selected from the group consisting of fluorosurfactants, a PEGylated rapeseedamide, glycerth 2-fatty acid esters, fatty acidamidoalkylenedi(alkyl)amines, and mixtures thereof comprising incorporating into such product at least one member selected from the group consisting of iodo propynyl butyl carbamate, gluconic acid, citric acid, parabens, antioxidants, alcohols, methyl-dibromo glutaronitrile, sodium hydroxymethyl glycinate, aliphatic dibrominated diol, methylchlorothiazolidinone, , and mixtures thereof, methylisothiazolidinone, chlorophenesin, sodium sulfite, salicyclic acid, triclosan, organic acids or salts thereof, formaldehyde donors, sodium erythorbate, quaternary ammonium compounds.

7. A method of improving the effectiveness of an active agent in a cleaning product containing an antimicrobial or a preservative and containing at least one member selected from the group consisting of iodo propynyl butyl carbamate, gluconic acid, citric acid, parabens, antioxidants, alcohols, methyl-dibromo glutaronitrile, sodium hydroxymethyl glycinate, aliphatic dibrominated diol, methylchlorothiazolidinone, , and mixtures thereof, methylisothiazolidinone, chlorophenesin, sodium sulfite, salicyclic acid, triclosan, organic acids or salts thereof, formaldehyde donors, sodium erythorbate, quaternary ammonium compounds comprising incorporating into said product at least one member selected from at least one member of the group consisting of fluorosurfactants, a PEGylated rapeseedamide, glycerth 2-fatty acid esters, fatty acidamidoalkylenedi(alkyl)amines, and mixtures thereof

8. A composition resulting from the method of claim 5.

9. A composition resulting from the method of claim 6.

10. A composition resulting from the method of claim 7.

11. A composition comprising the composition of claim 1 and at least one additional ingredient selected from the group of typical components of products selected from the group consisting of personal care products, industrial cleansers, household cleansers, and topical medicinal preparations.

12. A method of providing antibacterial activity to a composition comprising incorporating into said composition an antibacterially effective amount of at least one member selected from the group consisting of fluorosurfactants, a PEGylated rapeseedamide, glycerth 2-fatty acid esters, fatty acidamidoalkylenedi(alkyl)amines, and mixtures thereof.

13. A method of improving the antibacterially effectiveness of a product comprising incorporating therein at least one member selected from the group consisting of fluorosurfactants, a PEGylated rapeseedamide, glycerth 2-fatty acid esters, fatty acidamidoalkylenedi(alkyl)amines, and mixtures thereof, and optionally lactic acid.

14. The combination of claim 1 wherein said first component comprises from about 0.01 to about 100% of the weight of said combination.

15. The combination of claim 1 wherein said first component comprises from about 1% to about 80% of the total weight of said combination.

16. The combination of claim 1 wherein said first component comprises from about 0.0001 to about 4% of the total weight of said combination.

17. The combination of claim 1 wherein said first component comprises from about 0.0001 to about 2% of the total weight of said combination.
